(19) United States
(12) Patent Application Publication
(10) Pub. No.: US 2024/0335153 A1
(43) Pub. Date: Oct. 10, 2024

(54) ELECTROCARDIAC SIGNAL ANALYSIS DEVICE

(71) Applicant: MAXELL, LTD., Kyoto (JP)

(72) Inventors: Ayako SHINTANI, Kyoto (JP); Kazuaki SHIMADA, Kyoto (JP); Satoshi MATSUNUMA, Kyoto (JP); Reiji HATTORI, Kasuga-shi (JP)

(21) Appl. No.: 18/293,888

(22) PCT Filed: Jun. 17, 2022
(86) PCT No.: PCT/JP2022/024396

§ 371 Date: Jan. 31, 2024

(30) Foreign Application Priority Data
Aug. 2, 2021 (JP) 2021-126741

(51) Int. Cl. A61B 5/352; A61B 5/00; A61B 5/256

(52) U.S. Cl. CPC A61B 5/352; A61B 5/256; A61B 5/4035; A61B 5/7217; A61B 2503/20

(57) ABSTRACT

An electrocardiac signal analysis device measures an electrocardiac signal of a subject in a daily environment such as an office. The electrocardiac signal is analyzed by a plurality of methods, contributing to accurate evaluation of a health condition of the subject. A measurement unit includes a pair of detection electrodes of a capacitive coupling type that detect a heart rate of a subject in a non-contact state and output the heart rate as primary signals. A pair of active guard circuits reduce noise in the primary signals and output secondary signals. A potential difference of the secondary signals is amplified and output as an electrocardiac signal. A feedback electrode is configured to remove an influence of an in-phase signal of the secondary signals. An analysis unit linearly analyzes the electrocardiac signal to calculate an autonomic nerve index, and nonlinearly analyzes the electrocardiac signal to calculate a Lyapunov exponent.

### TECHNICAL FIELD

The present invention relates to an electrocardiac signal analysis device measures and analyzes an electrocardiac signal of a subject, for example, an employee working in an office. The analysis result of the electrocardiac signal by the present device can be used to evaluate the health condition, the degree of fatigue, stress, external fitness, and the like of the subject.

### BACKGROUND ART

In recent years, with an increase in health awareness, there is an increasing interest in preventive medicine for preventing diseases from occurring, and there is an increasing demand for a system for daily monitoring of physical and mental health. There is a demand for a technology for routinely measuring and analyzing biological information in various environments, such as ordinary homes and offices, in addition to medical related institutions. In particular, in an office environment, there is an increasing need to measure and analyze biological information of an employee who is working to use the information for maintaining health of the employee, and to detect a stress state at an early stage.

As a related art document related to measurement and analysis of biological information, for example, Patent Literature 1 can be cited. Patent Literature 1 discloses a sensor device that measures biological information of a subject, an evaluation device that evaluates a state of autonomic nerve of the subject on the basis of the obtained biological information, and the like. The sensor device includes a heart rate sensor that acquires heart rate information as biological information, and the sensor includes, for example, a pair of detection electrodes that come into contact with the surface of the body of the subject. The evaluation device calculates an RRI that is an interval between R waves from the obtained heart rate information, performs frequency analysis on the equal interval time-series data of the RRI by using, for example, fast Fourier transform, calculates a ratio of a low frequency component LF to a high frequency component HF of heart rate variability, that is, LF/HF, or the like as an autonomic nerve indicator, and evaluates the state of autonomic nerve of the subject on the basis of the index.

### CITATIONS LIST

### Patent Literature

Patent Literature 1: JP 2019-30389 A

### SUMMARY OF INVENTION

### Technical Problems

Patent Literature 1 exemplifies detection electrodes that come into contact with a surface of a body of a subject as a heart rate sensor. The wearing of this type of electrodes for a long time not only causes skin rash or metal allergy, but also gives discomfort or a sense of restraint to a subject, and the stress due to these troubles may affect the electrocardiac signal. Therefore, such detection electrodes are suitable for a situation such as a medical checkup in which an electrocardiac signal is temporarily measured, but are not suitable for daily measurement of an electrocardiac signal which is a main object of the present invention.

In addition, in the daily environment, various environmental noises exist unlike a hospital or the like in which the measurement environment of the electrocardiac signal is well-equipped. In particular, since there are many personal computers and other electric devices in an office environment, there are many hum noises derived from a commercial power supply (50 Hz or 60 Hz). When large environmental noise is mixed in the electrocardiac signal, the obtained electrocardiac signal may be inappropriate, and subsequent analysis may be difficult. Therefore, it is essential to reduce the environmental noise.

Furthermore, the present inventor found that the health condition, the fatigue level, stress, external fitness, and the like of a subject can be more accurately evaluated if an electrocardiac signal can be analyzed by a method different from the conventional method to obtain an analysis result different from a publicly known autonomic nerve index such as LF/HF, and completed the present invention.

An object of the present invention is to provide an electrocardiac signal analysis device that is capable of safely and accurately measuring an electrocardiac signal of a subject in a daily environment such as an office, obtaining a high-quality electrocardiac signal with less noise, and analyzing the electrocardiac signal by a plurality of methods, and that can contribute to accurate evaluation of a health condition or the like of the subject.

### Solutions to Problems

The present invention is directed to an electrocardiac signal analysis device including a measurement unit 1 that detects a heart rate of a subject and outputs an electrocardiac signal, and an analysis unit 2 that analyzes the electrocardiac signal obtained from the measurement unit 1. The measurement unit 1 includes a pair of detection electrodes 6, 6 of a capacitive coupling type that detect a heart rate of a subject in a non-contact state and output the heart rate as primary signals, a pair of active guard circuits 7, 7 that reduce noise included in the primary signals and output secondary signals, an amplification means 8 that amplifies a potential difference of the secondary signals and outputs an electrocardiac signal, and a feedback electrode 33 that is configured to remove an influence of an in-phase signal of the secondary signals. The analysis unit 2 includes a linear analytical means 3 that linearly analyzes the electrocardiac signal to calculate an autonomic nerve index, and a non-linear analytical means 4 that nonlinearly analyzes the electrocardiac signal to calculate a Lyapunov exponent.

The measurement unit 1 includes a high-pass filter 13 and a low-pass filter 14 that remove noise included in the electrocardiac signal amplified by the amplification means 8.

The amplification means 8 includes a first amplifier 11 that receives the secondary signals output from the active guard circuits 7, 7, and a second amplifier 12 that further amplifies the signal amplified by the first amplifier 11, and the high-pass filter 13 and the low-pass filter 14 are disposed between the first amplifier 11 and the second amplifier 12.

The linear analytical means 3 linearly analyzes a variation in an RRI that is an interval between R waves in the electrocardiac signal and calculates a ratio of a low frequency component LF to a high frequency component HF of a heart rate variability as the autonomic nerve index.

The non-linear analytical means 4 performs a chaotic analysis on a variation in an electrocardiac signal or in an RRI that is an interval between R waves in the electrocardiac signal to calculate a Lyapunov exponent.

Each active guard circuit 7 includes a guard electrode 18 paired with the detection electrode 6, and the detection electrode 6 and the guard electrode 18 are joined via an insulating layer 23 to be integrated as an electrode unit 28.

An entire of the electrode unit 28 including the detection electrode 6 and the guard electrode 18 is made of a flexible material.

### Advantageous Effects of Invention

In the electrocardiac signal analysis device according to the present invention, the heart rate of the subject is detected in a non-contact state by the detection electrode 6 of a capacitive coupling type. According to this, it is possible to safely detect the heart rate of the subject without causing skin rash or metal allergy which are concerned when the electrodes are directly worn on the body for a long time. In addition, it is possible to greatly reduce the sense of discomfort and the sense of restraint given to the subject by the wearing of the electrode, suppress the influence on the electrocardiac signal due to such a stress, and obtain an accurate electrocardiac signal. In addition, in the present invention, since the active guard circuit 7 that reduces noise included in the primary signal output from the detection electrode 6 is provided, it is possible to obtain a high-quality electrocardiac signal with less noise even when there are many noises, such as hum noise, around the subject. With the measurement unit 1 of the present invention including the detection electrode 6 and the active guard circuit 7 described above, it is possible to safely and accurately measure the electrocardiac signal of the subject in a daily environment such as an office and obtain a high-quality electrocardiac signal sufficient for the subsequent analysis by the analysis unit 2.

Furthermore, in the present invention, the analysis unit 2 that analyzes the electrocardiac signal includes the non-linear analytical means 4 that nonlinearly analyzes the electrocardiac signal, in addition to the linear analytical means 3 that linearly analyzes the electrocardiac signal as in a conventional manner. The advantage of non-linear analysis is that information that cannot be handled by the linear analysis can be handled. While it has been known that an electrocardiac signal has periodicity, it is found that “fluctuation” which has been considered as variation is a nonlinear phenomenon, for example. That is, an electrocardiac signal includes a nonlinear phenomenon. According to the findings by the present inventor, a Lyapunov exponent is useful as an index of the fitness of a subject to an external stimulus. With the analysis unit 2 of the present invention that performs non-linear analysis in addition to linear analysis, it is possible to contribute to more accurate evaluation of the health condition, the degree of fatigue, stress, external fitness, and the like of a subject, as compared with a conventional evaluation method that performs only linear analysis.

When the high-pass filter 13 and the low-pass filter 14 that remove noise included in the electrocardiac signal amplified by the amplification means 8 are provided, it is possible to obtain a clear electrocardiac signal from which noise is removed, the noise hindering the analysis by the analysis unit 2.

When the high-pass filter 13 and the low-pass filter 14 are disposed between the first amplifier 11 and the second amplifier 12 constituting the amplification means 8, the noise is removed by the filters 13 and 14 before being further amplified by the second amplifier 12, and the electrocardiac signal can be cleared.

When the detection electrode 6 and the guard electrode 18 are joined to each other via the insulating layer 23 to be integrated as the electrode unit 28, for example, the wearing of electrodes to the subject can be easily performed, as compared with a case where both electrodes are separated.

When the entire of the electrode unit 28 including the detection electrode 6 and the guard electrode 18 is made of a flexible material, the adhesive property of the electrode unit 28 to the subject is improved, and the electrocardiac signal can be stably measured.

### BRIEF DESCRIPTION OF DRAWINGS

FIG. 1 is a block diagram illustrating an entire of an electrocardiac signal analysis device according to an embodiment of the present invention.

FIG. 2 is a schematic configuration diagram of a measurement unit of an electrocardiac signal.

FIGS. 3A and 3B are sectional views of an electrode unit and a coaxial cable constituting the measurement unit.

FIG. 4A is an electrocardiogram output by the measurement unit of the present embodiment, and FIG. 4B is an electrocardiogram in a case where an active guard circuit is omitted.

FIG. 5A is an external view of a wearing belt to which the electrode unit is attached, and FIG. 5B is an explanatory diagram of a wearing method of the wearing belt.

FIG. 6 is a diagram illustrating a waveform of an electrocardiac signal to be analyzed.

FIGS. 7A and 7B include explanatory diagrams related to linear analysis of an electrocardiac signal, in which FIG. 7A illustrates equal interval time-series data of a heart rate interval, and FIG. 7B illustrates power spectral densities of a low frequency component and a high frequency component of heart rate variability.

FIG. 8 is an explanatory diagram related to non-linear analysis of an electrocardiac signal, and illustrates a procedure of obtaining an attractor by creating multidimensional time series data of heart rate intervals.

FIG. 9 is a scatter diagram obtained by performing a stress load test (task) on a subject, in which autonomic nerve indexes before and during the task are taken on a horizontal axis and a Lyapunov exponent is taken on a vertical axis.

FIG. 10 is a scatter diagram, in which autonomic nerve indexes before, during, and after the task are taken on a horizontal axis and a Lyapunov exponent is taken on a vertical axis.

### DESCRIPTION OF EMBODIMENT

### Embodiment

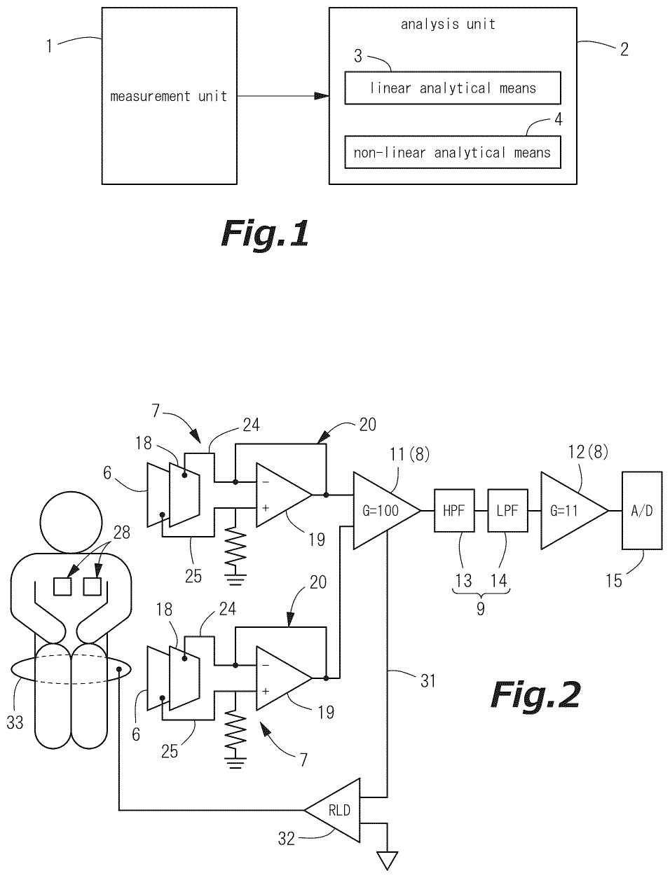

FIGS. 1 to 10 illustrate an embodiment of an electrocardiac signal analysis device (hereinafter simply referred to as analysis device) according to the present invention. As illustrated in FIG. 1, the electrocardiac signal analysis device includes a measurement unit 1 that detects a heart rate of a subject and outputs an electrocardiac signal, and an analysis unit 2 that analyzes the electrocardiac signal obtained from the measurement unit 1. The analysis unit 2 includes a linear analytical means 3 that linearly analyzes the electrocardiac signal to calculate an autonomic nerve index, and a non-linear analytical means 4 that nonlinearly analyzes the electrocardiac signal to calculate a Lyapunov exponent. Details of each of the analytical means 3 and 4 will be described later.

As illustrated in FIG. 2, the measurement unit 1 includes the pair of detection electrodes 6, 6 that detect a heart rate of a subject and output the heart rate as primary signals, the pair of active guard circuits 7, 7 that reduce environmental noise included in the primary signals and output secondary signals, an amplification means 8 that amplifies a potential difference of the secondary signals and outputs an electrocardiac signal, an analog filter 9 that removes line noise and interference noise from the electrocardiac signal, and the like. The amplification means 8 includes a first amplifier 11 that receives the outputs of the active guard circuits 7, 7, that is, the secondary signal, and a second amplifier 12 that further amplifies the signal amplified by the first amplifier 11. The analog filter 9 includes a high-pass filter 13 and a low-pass filter 14 disposed in series between the amplifiers 11 and 12. Both the amplifiers 11 and 12 include operational amplifiers, and the amplification factor of the first amplifier 11 is 100 and the amplification factor of the second amplifier 12 is 11. The electrocardiac signal amplified 1100 times through both the amplifiers 11 and 12 is digitally converted by an analog-digital converter 15 and then sent to the analysis unit 2.

When the measurement unit 1 is used in a daily environment such as an office, various environmental noises, such as hum noise, derived from a commercial power source are likely to be mixed in the primary signal output from the detection electrode 6. In order to reduce this environmental noise, the active guard circuit 7 is provided corresponding to each detection electrode 6. The active guard circuit 7 includes a guard electrode 18 paired with the detection electrode 6, a voltage follower 20 using an operational amplifier 19 having an amplification factor of 1, and a coaxial cable 21 connecting both electrodes 6, 18 to the voltage follower 20.

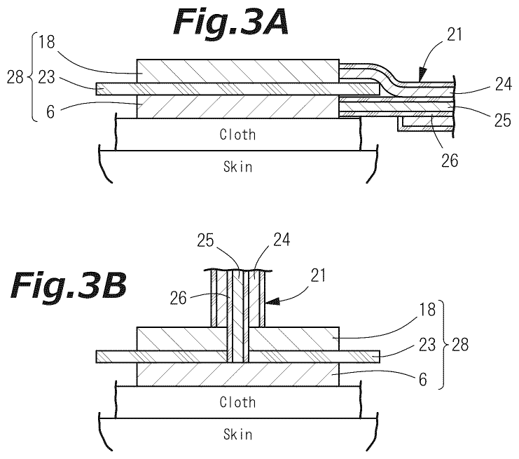

As illustrated in FIGS. 3A and 3B, the guard electrode 18 is bonded to the back surface (the back surface of the surface facing the subject) of the detection electrode 6 via the insulating layer 23, and is connected to an inverting input terminal (−) of the operational amplifier 19 by an outer conductor (shield) 24 of the coaxial cable 21. The detection electrode 6 is connected to a non-inverting input terminal (+) of the operational amplifier 19 by an inner conductor 25 of the coaxial cable 21. At the end portion of the coaxial cable 21 in the side on the electrodes 6, 18, the conductors 24, 25 may be connected to peripheries of the electrodes 18, 6 as illustrated in FIG. 3A, or the inner conductor 25 may penetrate the guard electrode 18 and the insulating layer 23 together with an insulating cylinder 26 covering the inner conductor 25 and may be connected to the detection electrode 6 as illustrated in FIG. 3B. Output terminals of the operational amplifiers 19 are connected to input terminals of the first amplifier 11 (the operational amplifier 19 of one active guard circuit 7 is connected to an inverting input terminal, and the operational amplifier 19 of the other active guard circuit 7 is connected to a non-inverting input terminal), and are connected (fed back) to the inverting input terminals of the operational amplifiers 19 via outer conductors 24. That is, the output terminal and the inverting input terminal of the operational amplifier 19 have the same potential. With the above configuration, the secondary signal in which environmental noise is reduced from the primary signal input to the non-inverting input terminal is output from the output terminal of the operational amplifier 19.

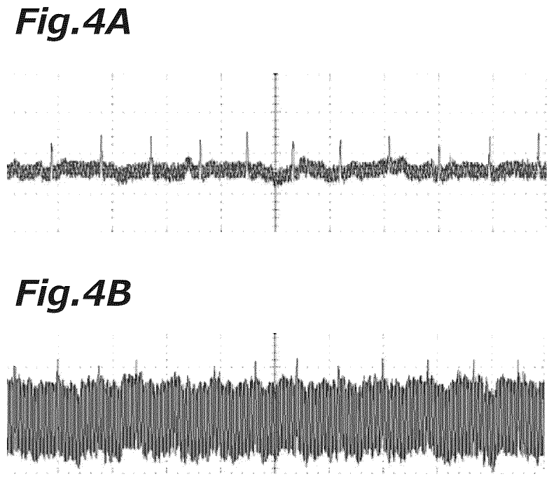

FIG. 4A illustrates an electrocardiogram output by the measurement unit 1 of the present embodiment including the active guard circuits 7, and FIG. 4B illustrates an electrocardiogram in a case where the active guard circuits 7 are not included, as a comparison target. As is clear from the comparison between the two diagrams, the active guards 7 according to the present embodiment are extremely useful for reducing environmental noise.

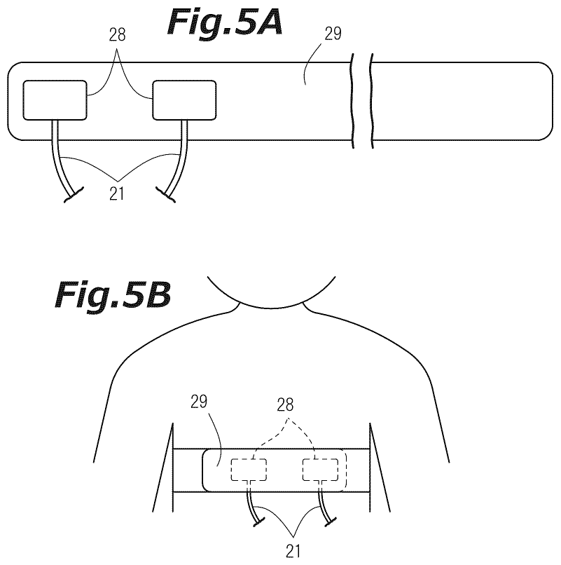

The detection electrode 6 and the guard electrode 18 are joined via the insulating layer 23 to constitute the electrode unit 28. Each of the electrode units 28 is worn near the heart of the subject, specifically, on the front surface side of a front body of clothing (insulator) such as underwear put on the upper body of the subject with the detection electrode 6 facing the subject. The wearing means is arbitrary, but for example, as illustrated in FIGS. 5A and 5B, a wearing belt 29 in which two electrode units 28 are laterally adjacent to each other and attached can be wound around and attached to the subject from above the clothing. In this wearing state, clothing is interposed between the detection electrode 6 and the skin of the subject. That is, each detection electrode 6 is arranged in a non-contact state with the subject to constitute a capacitive coupling type electrode. The capacitive coupling type electrode forms a capacitor between an electric signal source (heart) inside a living body and a metal plate (detection electrode 6) outside the living body, an electric signal in the living body being extracted from the metal plate outside the living body in a non-contact state.

In the present embodiment, the detection electrode 6 and the guard electrode 18 are formed of a rectangular sheet-like conductive foam having the same shape, and the insulating layer 23 is formed of an insulating urethane foam slightly larger than both electrodes 6, 18. If the entire of the electrode unit 28 is made of a flexible material, the adhesive property of the electrode unit 28 to the subject is improved, and the electrocardiac signal can be stably measured. The materials of the detection electrode 6 and the guard electrode 18 are not limited to the conductive foam, and for example, both electrodes 6, 18 can be formed of a thin metal plate made of stainless steel.

As illustrated in FIG. 2, the first amplifier 11 includes an inverting output means 31 that inverts and outputs the in-phase signal of the secondary signals input from the active guard circuits 7. The inverting output means 31 is connected to one input terminal of a feedback amplifier 32, and the other input terminal of the feedback amplifier 32 is set to the reference potential. An output terminal of the feedback amplifier 32 is connected to the feedback electrode 33 provided on a seat surface of a chair on which the subject sits. In the present embodiment, the feedback electrode 33 is made of conductive rubber. By the function of the feedback electrode 33 or the like, the influence of the in-phase signal of the secondary signals can be removed.

As described above, in the measurement unit 1 of the analysis device according to the present embodiment, the heart rate of the subject is detected in a non-contact state by the detection electrode 6 of a capacitive coupling type. According to this, it is possible to safely detect the heart rate of the subject without causing skin rash or metal allergy which are concerned when the electrodes are directly worn on the body for a long time. In addition, it is possible to greatly reduce the sense of discomfort and the sense of restraint given to the subject by the wearing of the electrode, suppress the influence on the electrocardiac signal due to such a stress, and obtain an accurate electrocardiac signal. In addition, in the present embodiment, since the active guard circuit 7 that reduces noise included in the primary signal output from the detection electrode 6 is provided, it is possible to obtain a high-quality electrocardiac signal with less noise even when there are many noises, such as hum noise, around the subject. With the measurement unit 1 of the present embodiment including the detection electrode 6 and the active guard circuit 7 described above, it is possible to safely and accurately measure the electrocardiac signal of the subject in a daily environment such as an office and obtain a high-quality electrocardiac signal sufficient for the subsequent analysis by the analysis unit 2.

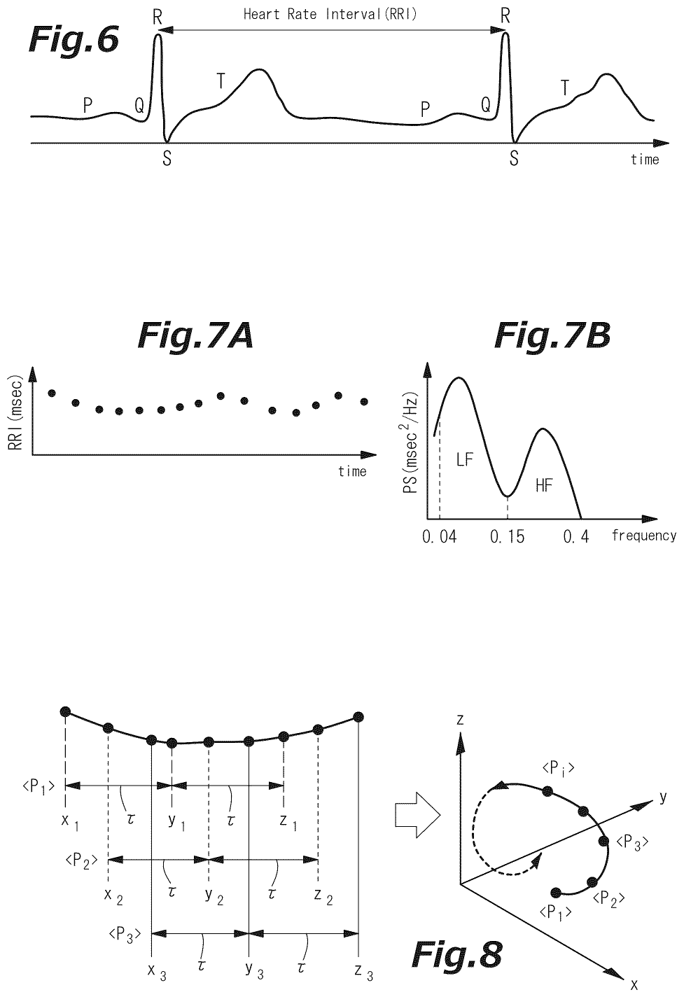

The analysis unit 2 that has obtained the electrocardiac signal from the measurement unit 1 simultaneously calculates an autonomic nerve index and a Lyapunov exponent by the linear analytical means 3 and the non-linear analytical means 4. First, the linear analytical means 3 calculates an RRI (heart rate interval) that is an interval between R waves from the electrocardiac signal illustrated in FIG. 6, and linearly analyzes the variation of RRI. Specifically, frequency analysis is performed on the equal interval time-series data of the RRI (see FIG. 7A) by using fast Fourier transform, the power spectral density (see FIG. 7B) of the low frequency component LF (0.04 Hz to 0.15 Hz) and the high frequency component HF (0.15 Hz to 0.4 Hz) of the heart rate variability is obtained, and the ratio of the low frequency component LF to the high frequency component HF, that is, LF/HF is calculated as a stress index indicating the degree of activity of sympathetic nerve.

In a low stress state in which the parasympathetic nerve in the autonomic nerve is activated, both the HF component and the LF component appear, but in a high stress state in which the sympathetic nerve is activated, the LF component appears while the HF component decreases. That is, in the low stress state, the value of LF/HF becomes small because the HF component becomes relatively large, and conversely, in the high stress state, the value of LF/HF becomes large because the LF component becomes larger than the HF component.

The non-linear analytical means 4 nonlinearly analyzes a variation in an RRI (heart rate interval), specifically, performs a chaotic analysis to calculate a Lyapunov exponent. First, as illustrated in FIG. 8, an attractor is obtained by creating multidimensional time series data of the RRI (the dimension is six in the present embodiment, and the dimension is three for simplification in FIG. 8). That is, coordinates Pᵢ(xᵢ, yᵢ, zᵢ) are sequentially plotted from P₁ in a multidimensional space. When the value of RRI is R (t), xᵢ=R (i), yᵢ=R (i+τ), zᵢ=R (i+2τ), and τ=1 (second) in the present embodiment.

A quantized chaotic property of a trajectory in the attractor is the Lyapunov exponent. It is possible to calculate the Lyapunov exponent by calculating the time variation amount of the attractor expanding exponentially to infinity. If this Lyapunov exponent is positive, it can be said that the trajectory has a chaotic property, and it can be said that, as the value is larger, the trajectory is more complicated and the fluctuation increases. According to the findings by the present inventor, a Lyapunov exponent is useful as an index of the fitness of a subject to an external stimulus and can be an index of a concentration degree or a stress state.

The advantage of non-linear analysis such as a chaotic analysis is that information that cannot be handled by the linear analysis can be handled. While it has been known that an electrocardiac signal has periodicity, it is found that “fluctuation” which has been considered as variation is a nonlinear phenomenon, for example. That is, an electrocardiac signal includes a nonlinear phenomenon. With the analysis unit 2 of the present embodiment that performs non-linear analysis in addition to linear analysis, it is possible to contribute to more accurate evaluation of the health condition, the degree of fatigue, stress, external fitness, and the like of a subject, as compared with a conventional evaluation method that performs only linear analysis. As described above, the analysis device according to the present embodiment can contribute to Goal 3 (ensure healthy lives and promote well-being for all at all ages) of the sustainable development goals (SDGs) advocated by the United Nations.

Next, a stress load experiment in which stress is applied to a subject to measure and analyze an electrocardiac signal will be described. Here, a stress load task was performed on a female subject in her twenties, electrocardiac signals during and before the task were measured, and an autonomic nerve index (LF/HF) and a Lyapunov exponent of each were calculated. The execution time length of the task and the measurement time lengths before and after the task were each 200 seconds. As the stress load task, the Stroop color-word test known as a neuropsychological test for measuring the function of suppressing attention and interference of the frontal lobe was performed.

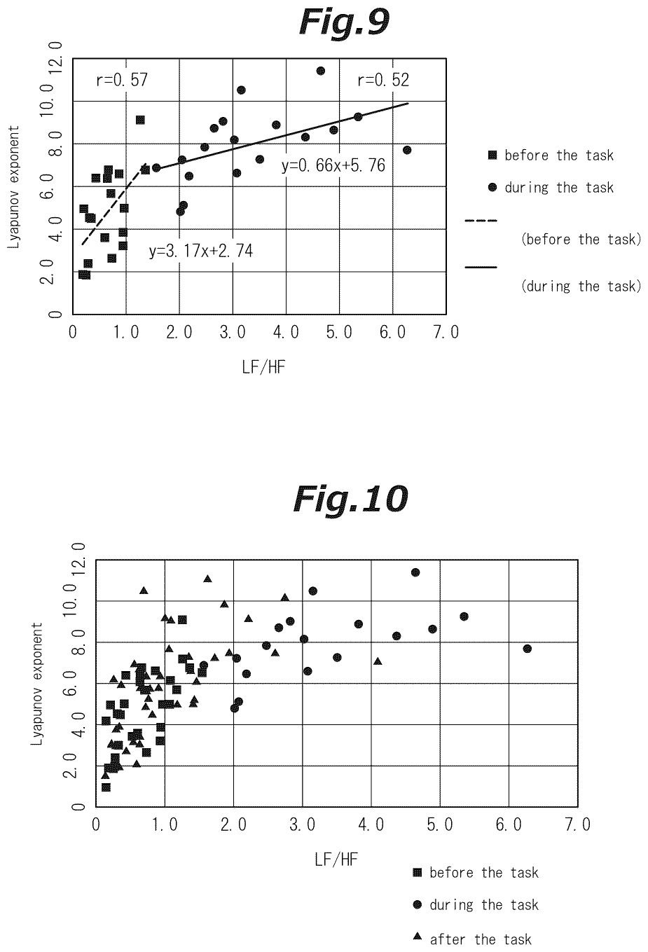

FIG. 9 is a scatter diagram, in which values of LF/HF before and during the task are taken on a horizontal axis and a Lyapunov exponent is taken on a vertical axis. From this scatter diagram, it can be seen that both the LF/HF and the Lyapunov exponent become larger during the task than before the task, and the LF/HF and the Lyapunov exponent have a certain correlation (correlation coefficient before task=0.57, correlation coefficient during task=0.52). However, the proportionality factor of a linear approximation line is relatively large before the task and relatively small during the task (proportionality factor before task=3.17, proportionality factor during task=0.66). According to the above results, as compared with the LF/HF, it is considered that the Lyapunov exponent possibly reflects even a small influence, being affected to a subject from the environment, to its value.

FIG. 10 is a scatter diagram of FIG. 9 to which values after the task are added. From the scatter diagram, it can be seen that the value of LF/HF decreases relatively quickly after the task (returns to the value before the task), whereas the value of the Lyapunov exponent tends not to decrease (maintains the value during the task). From this, as compared with the LF/HF, it is considered that the Lyapunov exponent is suitable for evaluation of relatively long-term stress.

### REFERENCE SIGNS LIST

1 Measurement Unit
2 Analysis Unit
3 Linear Analytical Means
4 Non-linear Analytical Means
6 Detection Electrode
7 Active Guard Circuit
8 Amplification Means
11 First Amplifier
12 Second Amplifier
13 High-pass Filter
14 Low-pass Filter
18 Guard Electrode
23 Insulating Layer
28 Electrode Unit
33 Feedback Electrode

1.-7. (canceled)

8. An electrocardiac signal analysis device comprising:
a measurement unit that detects a heart rate of a subject and outputs an electrocardiac signal; and
an analysis unit that analyzes the electrocardiac signal obtained from the measurement unit,
wherein the measurement unit includes
a pair of detection electrodes of a capacitive coupling type that detect a heart rate of a subject in a non-contact state and output the heart rate as primary signals,
a pair of active guard circuits that reduce noise included in the primary signals and output secondary signals,
an amplification means that amplifies a potential difference of the secondary signals and outputs an electrocardiac signal, and
a feedback electrode that is configured to remove an influence of an in-phase signal of the secondary signals, and
the analysis unit includes
a linear analytical means that linearly analyzes the electrocardiac signal to calculate an autonomic nerve index, and
a non-linear analytical means that nonlinearly analyzes the electrocardiac signal to calculate a Lyapunov exponent.

9. The electrocardiac signal analysis device according to claim 8, wherein the measurement unit includes a high-pass filter and a low-pass filter that remove noise included in the electrocardiac signal amplified by the amplification means.

10. The electrocardiac signal analysis device according to claim 9, wherein
the amplification means includes
a first amplifier that receives the secondary signals output from the active guard circuits, and
a second amplifier that further amplifies the signal amplified by the first amplifier, and
the high-pass filter and the low-pass filter are disposed between the first amplifier and the second amplifier.

11. The electrocardiac signal analysis device according to claim 8, wherein the linear analytical means linearly analyzes a variation in an RRI that is an interval between R waves in the electrocardiac signal and calculates a ratio of a low frequency component (LF) to a high frequency component (HF) of a heart rate variability as the autonomic nerve index.

12. The electrocardiac signal analysis device according to claim 9, wherein the linear analytical means linearly analyzes a variation in an RRI that is an interval between R waves in the electrocardiac signal and calculates a ratio of a low frequency component (LF) to a high frequency component (HF) of a heart rate variability as the autonomic nerve index.

13. The electrocardiac signal analysis device according to claim 10, wherein the linear analytical means linearly analyzes a variation in an RRI that is an interval between R waves in the electrocardiac signal and calculates a ratio of a low frequency component (LF) to a high frequency component (HF) of a heart rate variability as the autonomic nerve index.

14. The electrocardiac signal analysis device according to claim 8, wherein the non-linear analytical means performs a chaotic analysis on a variation in an electrocardiac signal or in an RRI that is an interval between R waves in the electrocardiac signal to calculate a Lyapunov exponent.

15. The electrocardiac signal analysis device according to claim 9, wherein the non-linear analytical means performs a chaotic analysis on a variation in an electrocardiac signal or in an RRI that is an interval between R waves in the electrocardiac signal to calculate a Lyapunov exponent.

16. The electrocardiac signal analysis device according to claim 10, wherein the non-linear analytical means performs a chaotic analysis on a variation in an electrocardiac signal or in an RRI that is an interval between R waves in the electrocardiac signal to calculate a Lyapunov exponent.

17. The electrocardiac signal analysis device according to claim 10, wherein
each active guard circuit includes a guard electrode paired with the detection electrode, and
the detection electrode and the guard electrode are joined via an insulating layer to be integrated as an electrode unit.

18. The electrocardiac signal analysis device according to claim 17, wherein an entire of the electrode unit including the detection electrode and the guard electrode is made of a flexible material.
